(19) United States
(12) Patent Application Publication
(10) Pub. No.: US 2011/0236933 A1
(43) Pub. Date: Sep. 29, 2011

(54) RECOMBINANT EUKARYOTIC EXPRESSION PLASMID ENCODING pprI GENE OF DEINOCOCCUS RADIODURANS R1 AND ITS FUNCTIONS

(75) Inventors: Zhanshan Yang, Suzhou (CN); Na Li, Suzhou (CN); Tianchang Wang, Suzhou (CN); Tingting Chen, Suzhou (CN); Yongqin Zhang, Suzhou (CN)

(73) Assignee: SOOCHOW UNIVERSITY, Suzhou (CN)

(21) Appl. No.: 12/730,769

(22) Filed: Mar. 24, 2010

(51) Int. Cl. C12N 15/66; C12N 1/21; C12N 15/70

(52) U.S. Cl. 435/91.41; 435/252.33; 435/320.1

(57) ABSTRACT

The present invention concerns a novel recombinant eukaryotic expression plasmid pCMV-HA-pprI encoding the pprI gene isolated from Deinococcus radiodurans R1, the method for preparing pCMV-HA-pprI, and its expression in human 293T cells. The present invention also discloses the optimal method and process of pprI gene transfection by in vivo electroporation, and the radioprotective and therapeutic effects of the recombinant pCMV-HA-pprI on lethally irradiated mice.

### FIELD OF THE INVENTION

The present invention relates to a gene recombinant vector, specifically a recombinant eukaryotic expression plasmid encoding the pprI gene of Deinococcus radiodurans R1, a method of constructing the pCMV-HA-pprI eukaryotic vector and its effects in mitigating acute radiation injury in mice.

### BACKGROUND

With the development of nuclear science and technology, nuclear radiation has been widely used in national defense, industrial, agricultural and medical fields. The radioactive applications have brought great benefits to mankind. However, whether for peaceful or military applications, high-dose ionizing radiation can cause severe acute radiation injury (ARI) and a very high risk of mortality. The prevention or treatment of severe ARI is difficult and is an on-going field of research in international radioprotection.

Recently, with the rapid development and application of molecular biology and protein engineering science, cytokines such as G-CSF and GM-CSF have been used in clinical treatment of ARI. Our recent research shows that the expression of cytokines in irradiated tissues was significantly reduced and disrupted (X H Pan et al. Chinese j Radio Med. Prot. 2007, 27 (3): 219-222), which may be an important factor for severe ARI being difficult to treat effectively. Hence, it is very important for the repair of ARI that there is a continuous controllability release of the cytokines.

However, the clinical application of cytokines by hypodermic or intramuscular injection are limited due to its expense and limited biological half-life. Increasing the drug dose or injection frequency might cause intolerable side effects.

Deinococcus radiodurans (“D. radiodurans”) is an aerobic, non-spore forming, gram-positive quadruplex micrococcus. It was discovered and isolated by A W Anderson et al in 1956 from adulterated canned meat after X-ray radiation. It is one of the most radiation-resistant micrococci known so far. D. radiodurans has extraordinary resistance to ionizing radiation, ultraviolet light and other DNA damaging agents (W. Minton. J. Mol. Microbiol., 1994, 13:9-15), as such has attracted considerable research interest. Its capacity for repairing ionizing radiation-induced DNA double-strand breaks (DSBs) is quite exceptional; it can repair 150 DSBs per chromosome within a few hours of irradiation. D. radiodurans in the exponential growth phase is able to survive acute exposures to γ-irradiation exceeding 17 kGy without lethality or induced mutation. By comparison, this is over 200 times greater radioresistance than stationary-phase Escherichia coli (White O et al. Science, 1999, 286:1571-15771). Although the mechanisms of the extraordinary radioresistance in D. radiodurans remains poorly understood, current research indicates that this is probably related to its tight ring-like chromosomal structure (Levin-Zaidman S et al. Science, 2003, 299:1571-1577) and its efficient DNA DSBs repair capability (R. Battista et al. Trends Microbiol, 1999, 7:362-365; J. R. Battista et al. Curr Biol. 2000, 10:R204-R205). This is also probably linked to unusual concentrations of trace elements whereby manganese is abundant whilst iron is reduced in its cytosol.

pprI (inducer of pleiotropic proteins promoting DNA repair) is a newly identified regulatory protein from Deinococcus radiodurans. The length of the pprI gene is 987 bp, and it encodes for a protein with 328 amino acids whose molecular weight is about 37 KD. pprI is a responsible element for the extreme radioresistance of D. radiodurans, which stimulates transcription of the recA gene following exposure to ionizing radiation (A. M. Earl et al. J. Bacteriol. 2002, 184:6216-6224). It serves as a general switch for DNA repair and protection pathways via its regulatory function on the expression of downstream recA and pprA genes (Y. J. Hua et al. Biophy. Res. Commun. 2003, 306: 354-360; Gao G J et al. DNA Repair. 2003, 2: 1419-1427). The research results show that exogenous expression of PprI protein may promote DNA repair and enhance the radioresistance and oxidation resistance of E. coli.

D. radiodurans is a prokaryote and thus differs considerably from eukaryotes; their evolution having diverged very early on. Their differences are reflected in their gene composition, methods of protein expression, codon preference and so on. Therefore the expression of a prokaryotic gene in mammalian cells is almost impossible. Moreover, PprI protein has no homologous analogue in mammalian cells.

### SUMMARY OF THE INVENTION

The present invention provides a recombinant eukaryotic plasmid pCMV-HA-pprI encoding pprI gene from D. radiodurans R1. The PprI protain can be successfully expressed by transfecting the pCMV-HA-pprI into different mammalian species cells and confirmed by Western blotting. The present invention also provides the use of recombinant eukaryotic plasmid pCMV-HA-pprI, and shows the role of the recombinant plasmid in repairing acute radiation injury.

To achieve the above objective, the present invention provides a recombinant eukaryotic plasmid pCMV-HA-pprI that can encode pprI gene from D. radiodurans R1. And the recombinant plasmid pCMV-HA-pprI was transfected it into E. coli DH5α in order to conserve the plasmids. The E. coli DH5α containing the recombinant plasmid pCMV-HA-pprI was deposited in the China Center for Type Culture Collection (CCTCC). And the details of deposition information are listed below: the title of the depositary institution: China Center for Type Culture Collection (CCTCC); the address of the depositary institution: Wuhan University, China; the date of deposition: Dec. 11, 2008; the scientific name: Escherichia coli DH5α/pCMV-HA-pprI; the accession number of the deposit: CCTCC NO.: M208253.

The method for constructing said pCMV-HA-pprI eukaryotic vector comprising:
(1) Obtaining pprI gene by PCR amplification using isolated total genomic DNA from Deinococcus radiodurans R1 as templates, using the primer (5′-ATGCCCAGTGCCAACGTCAGCCCCCCTT-3′) as upstream primer, the primer (5′-TCACTGTGCAGCGTCCTGCGGCTCGTCC-3′) as downstream primer, purifying and detecting and quantifying the PCR products, obtaining PCR product pprI gene;
(2) Ligating the PCR product pprI gene into a sub-cloning vector pGEM-T, then transferring the ligated product pGEM-T-pprI into E. coli DH5α, and picking out the positive clones after incubation of the bacteria, then extracting and sequencing the recombinant vector pGEM-T-pprI;
(3) Obtaining pprI fragment by PCR amplification using the recombinant vector pGEM-T-pprI as templates, using the primer (5′-TCGAATTCCCAGTGCCAACGTCAGCCCCCCTTGC-3′) as upstream primer, the primer (5′-TTCTCGAGTTTCACTGTGCAGCGTCCTGCGGCTC-3′) as downstream primer, obtaining the PCR product pprI fragment;
(4) Digesting the PCR product pprI fragment and the pCMV-HA vector by enzymes EcoRI and XhoI, obtaining the digested product pprI fragment digested pCMV-HA vector, then purifying and ligating the digested product pprI fragment into the digested pCMV-HA vector to construct the recombinant plasmid pCMV-HA-pprI.

Specifically, the method of constructing E. coli DH5α containing the recombinant plasmid pCMV-HA-pprI includes the following steps:

(1) In order to clone the pprI coding region, PCR was carried out using the total genomic DNA of D. radiodurnas R1 with the following primers: forward primer: 5′-ATGCCCAGTGCCAACGTCAGCCCCCCTT-3′ and reverse primer: 5′-TCACTGTGCAGCGTCCTGCGGCTCGTCC-3′.

(2) The PCR product was ligated into the sub-cloning vector pGEM-T, the ligated product pGEM-T-pprI was transformed into E. coli DH5α, then the positive clones were picked out, and the plasmid pGEM-T-pprI was extracted and sequenced.

(3) PCR was finished using the recombinant vector pGEM-T-pprI as a template DNA with the following primers: 5′-TCGAATTCCCAGTGCCAACGTCAGCCCCCCTTGC-3′ and 5′-TTCTCGAGTTTCACTGTGCAGCGTCCTGCGGCTC-3′. The underlined sequences are restriction sites of EcoRI and XhoI respectively. Then the PCR products were separated by agarose gel electrophoresis to check whether the PCR had generated the anticipated DNA fragment.

(4) The purified PCR products and the pCMV-HA vector were digested respectively by EcoRI and XhoI. Then the pprI fragment was ligated into the pCMV-HA vector with mole ratio of 5:1. The recombinant vectors pCMV-HA-pprI were transfected into E. coli DH5α, and cultured on LB plates solidified with 1.5% agar and supplemented with 100 μg/ml of ampicillin. The positive clones were picked out from culture after 12 h. PCR amplification was carried on the different positive clones. Those clones containing the recombinant vectors pCMV-HA-pprI were selected out by agarose gel electrophoresis. The recombinant vectors pCMV-HA-pprI were extracted from the clones and sequenced. Those bacteria of the positive clones with the correct sequence were cultured and frozen down at −80° C.

In the above methods, the vector pCMV-HA is a pre-existing vector and its map is shown in FIG. 4. The multiple cloning site (MCS) and restriction sites in the pCMV-HA vector are in FIG. 5.

The present invention also includes an application of the recombinant eukaryotic expression vector pCMV-HA-pprI encoding the pprI gene to the preparation of PprI protein as a drug for conferring resistance to acute radiation injury.

The present invention also includes a gene therapy drug containing above-said recombinant vector pCMV-HA-pprI for the prevention or treatment of acute radiation injury.

The recombinant vectors pCMV-HA-pprI described in the present invention were conserved in E. coli DH5α. The method of extraction the plasmids is that the E. coli DH5α was cultured in 5 ml of LB liquid medium supplemented with 100 μg/ml of ampicillin, then the recombinant plasmids pCMV-HA-pprI were extracted using a small-scale preparation of purified plasmid DNA isolation kit after 12 h of shaking culture.

The advantages of the invention are: (1) The recombinant plasmid pCMV-HA-pprI was successfully constructed with two PCR primers and PprI protein could be expressed in mammalian cells, demonstrating that the prokaryotic pprI gene from D. radiodurans was successfully expressed in mammalian cells; and (2) The recombinant plasmid pCMV-HA-pprI in the present invention was transferred into mice muscle by electroporation in vivo. It showed significant effect on prevention and efficacy of treatment of acute radiation injury, and had a long biological half-life and few side effects.

### BRIEF DESCRIPTION OF THE DRAWINGS

FIG. 1. depicts the electrophoresis map of pprI segment in example 1, cloned from the total genomic DNA of D. radiodurnas R1 by PCR-amplification, where M denotes DL2000 Marker; Lane 1 denotes about 1 kb length of pprI gene.

FIG. 2. depicts the electrophoresis map of pprI segment in example 1, sub-cloned from different E. coli DH5α clones by PCR-amplification, where M denotes DL2000 Marker and Lane 1 to Lane 5 show PCR-amplification with different E. coli DH5α clones; and Lane 3 shows pprI segment sub-cloned by PCR-amplification from E. coli DH5α clones containing recombinant plasmid pCMV-HA-pprI.

FIG. 3. depicts the electrophoresis map of PprI protein expression in 293T cells detected by Western blotting in example 2, where 293T cells were transfected with the recombinant plasmid pCMV-HA-pprI and the existing vector pCMV-HA, respectively. The molecular weight of the PprI protein is about 37 KD.

FIG. 4. depicts the map of the existing vector pCMV-HA in example 1.

FIG. 5. depicts the multiple cloning site (MCS) and restriction sites in the pCMV-HA vector in example 1.

Photos. 6. depicts the histopathological changes of lung, liver, kidney and testis from irradiated mice in example 4, where PHOTO 6A shows the histopathological changes in lung in the radiation group; PHOTO 6B shows the histopathological changes in lung in the irradiated transgene group; PHOTO 6C shows the histopathological changes in liver in the radiation group; PHOTO 6D shows the histopathological changes in liver in the irradiated transgene group; PHOTOS 6E1 and 6E2 show the histopathological changes in kidney in the radiation group; PHOTOS 6F1 and 6F2 show the histopathological changes in kidney in the irradiated transgene group; PHOTO 6G shows the histopathological changes in testis in the radiation group; PHOTO 6H shows the histopathological changes in testis in the irradiated transgene group.

### DETAILED DESCRIPTION OF THE PREFERRED EMBODIMENTS

Materials: pGEM-T vector (Promega Biotec), pCMV-HA vector (Clontech), Lipofectinamine2000 (Invitrogen), DMEM Medium and Bovine Serum (GIBCO), HA-Tag Mouse mAb (Cell Signaling Technology), Anti-mouse IgG HRP-linked Antibody (Cell Signaling Technology), High Pure Plasmid Isolation Kit (Roche).

Cells culture: D. radiodurans R1 was grown in TGY broth medium (0.5% bacto-tryptone, 0.1% glucose, 0.3% bacto-yeast extract) at 30° C. with aeration. E. coli was grown at 37° C. in LB broth medium or on LB plates solidified with 1.5% agar and supplemented with 100 μg/ml of ampicillin when the positive clones are selected. E. coli was transformed by the modified CaCl₂ technique. 293T cells were cultured in DMEM high glucose medium supplemented with 10% bovine serum at 37° C. in an atmosphere of 5% CO₂.

The present invention is further illustrated by the following examples, which however, are not to be construed as limiting the scope of protection.

### Example 1

### Construction of Recombinant Plasmid pCMV-HA-pprI

(1) Clone of pprI Gene

Total genomic DNA of D. radiodurnas R1 is isolated by the method provided by Maniatis T et al. (Molecular cloning: A laboratory manual. 1989, 2nd Ed. New York: Cold Spring Harbor Laboratory Press), and clone primers are designed according to the genomic DNA sequence:

The forward primer:
5′-ATGCCCAGTGCCAACGTCAGCCCCCCTT-3′
The reverse primer:
5′-TCACTGTGCAGCGTCCTGCGGCTCGTCC-3′

PCR was carried out with the total genomic DNA of D. radiodurnas R1 as a template, and cycling conditions were as follows: 1 cycle of 5 min at 94° C., 35 cycles of 1 min at 94° C., 1 min at 54° C. and 1 min at 72° C., 1 cycle of 10 min at 72° C.

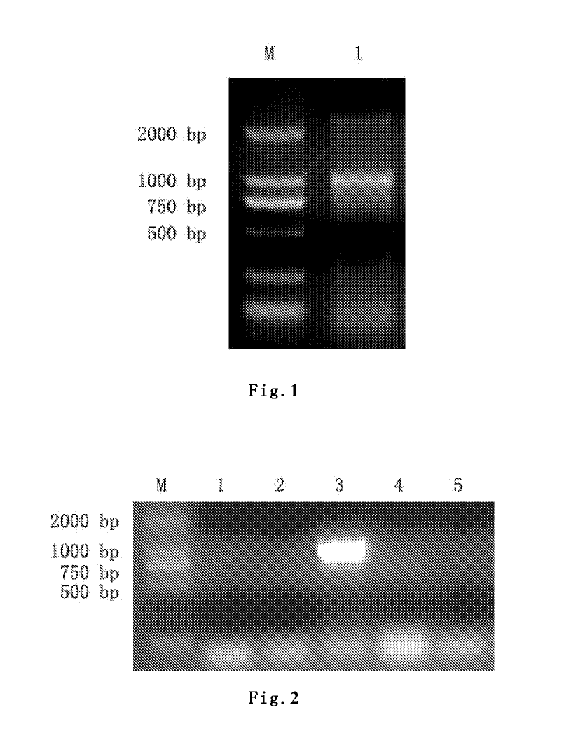

After chilling the reaction mixture, PCR products were detected by agarose gel electrophoresis, and purified by a purification kit and quantified. The target segment is about 1 kb (FIG. 1).

(2) The Construction of Recombinant Plasmid pCMV-HA-pprI

a. The Construction of pGEM-T-pprI

The PCR product was ligated into the sub-cloning vector pGEM-T, and the ligated product pGEM-T-pprI was transfected into E. coli DH5α. Then the positive clones were picked out and sequenced.

b. The Construction of pCMV-HA-pprI

The recombinant vector pGEM-T-pprI was subcloned into the eukaryotic expressing vector pCMV-HA using PCR-amplification with the following primers:

5′-TCGAATTCCCAGTGCCAACGTCAGCCCCCCTTGC-3′ and 5′-TTCTCGAGTTTCACTGTGCAGCGTCCTGCGGCTC-3′ containing restriction sites for EcoRI and XhoI (underlined sequences) respectively. The PCR product was digested with EcoRI and XhoI, and the fragment was then ligated into the pCMV-HA vector that had been predigested with the two enzymes. The recombinant plasmid pCMV-HA-pprI was transfected into host cells E. coli DH5α. The host cells E. coli DH5α were cultured on LB plates solidified with 1.5% agar and supplemented with 100 μg/ml of ampicillin. After a 12 hour incubation the positive clones were picked out. PCR-amplification was carried out from different positive clones so as to select out those positive clones containing recombinant plasmid pCMV-HA-pprI by agarose gel electrophoresis (FIG. 2). The recombinant plasmid was then isolated from the positive clones and sequenced, the results of forward sequence and reverse sequence are respectively shown in SEQ ID NO.1 and SEQ ID NO.2 of the attached Sequence Listing, and the sequences were certified by checking in NCBI (DR_{—}0167).

### Example 2

### Transfection and Expression of the pprI Gene

The recombinant plasmid pCMV-HA-pprI was transfected into the human embryonic kidney 293T cells, and expression of the pprI gene was identified by Western blotting.

1 ml of 293T cells at a density of 1×10⁵ cells/ml was put into a 35 mm culture dish, and incubated in DMEM medium (high glucose) supplemented with 10% bovine serum without antibiotics for about 18 hours. When the 293T cells were 70-80% confluent, the growth medium was changed to Optimen (GIBCO) medium without serum. 1 μg of pCMV-HA-pprI plasmid and 3 μl of lipofectamine2000 were transfected into the cells according to the manufacturer's instructions. At the same time 1 μg of vector pCMV-HA were transfected as control. After 4-6 hours incubation the growth medium was replaced with DMEM medium containing 10% bovine serum, and the cells were incubated at 37° C. for 24 h in a CO₂ incubator.

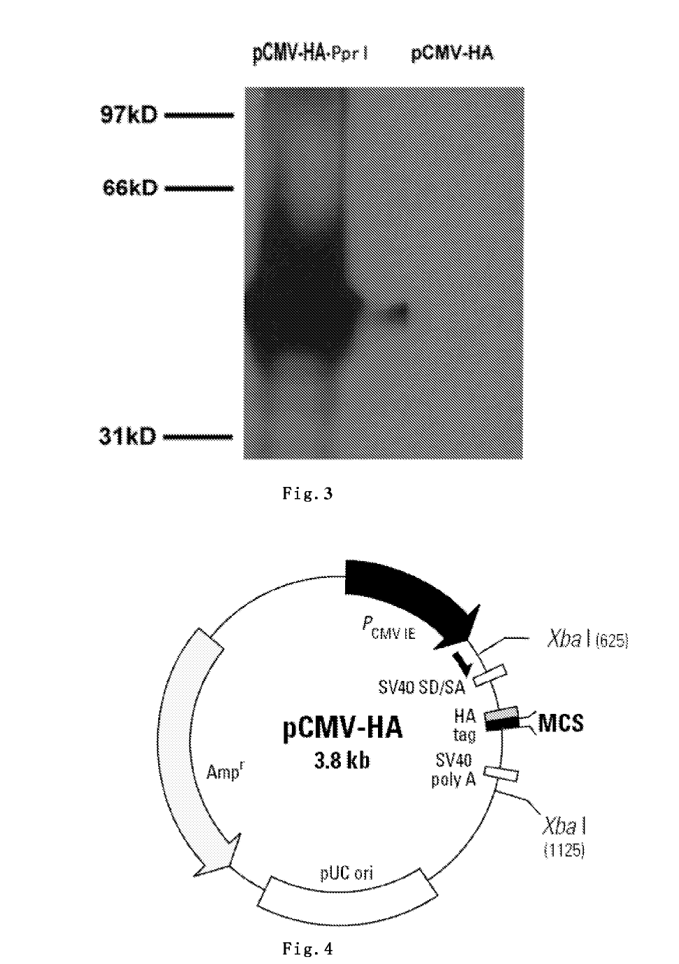

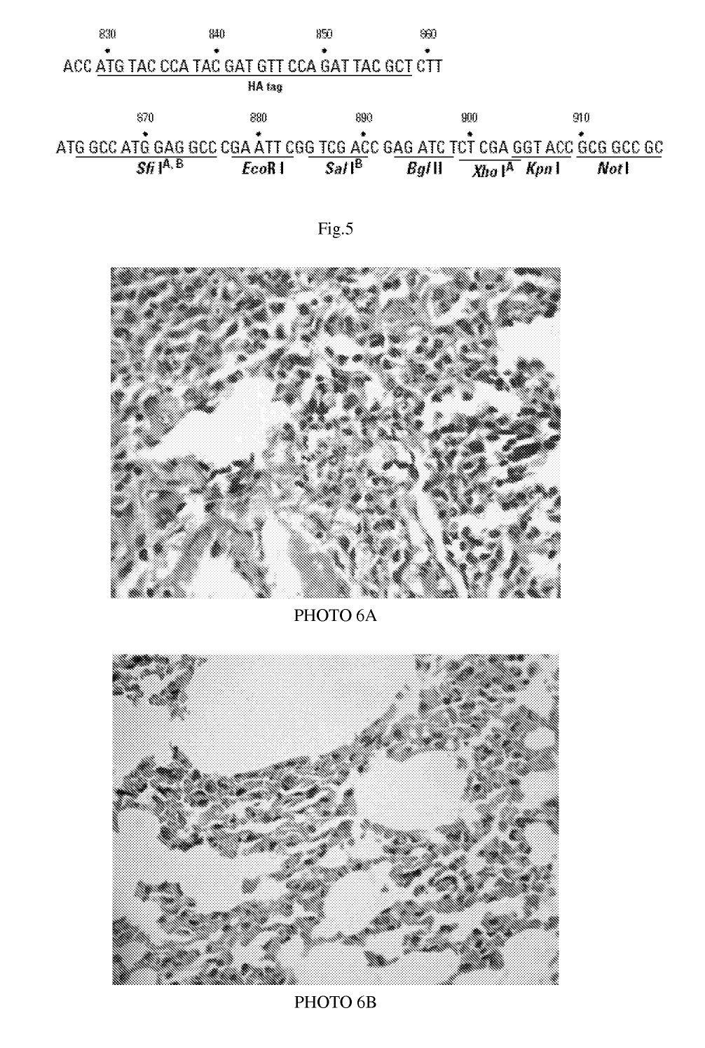

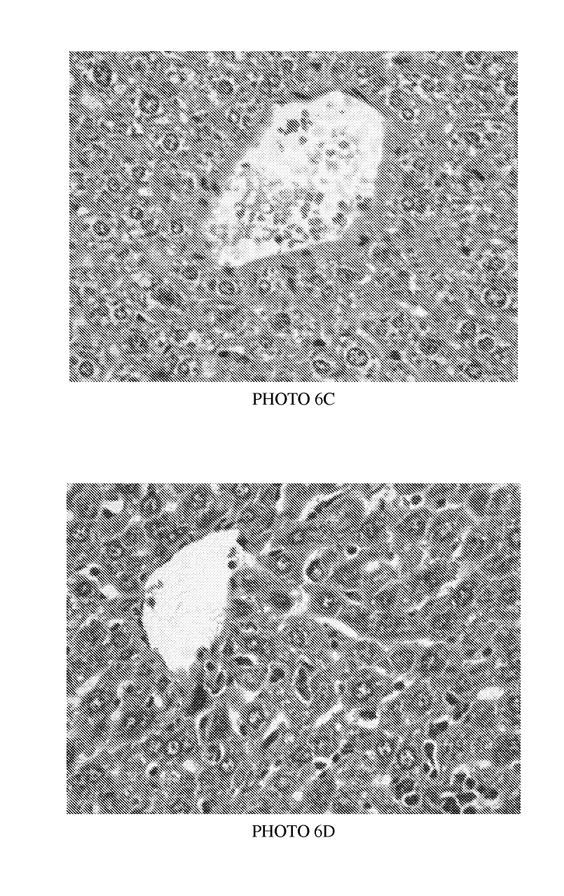

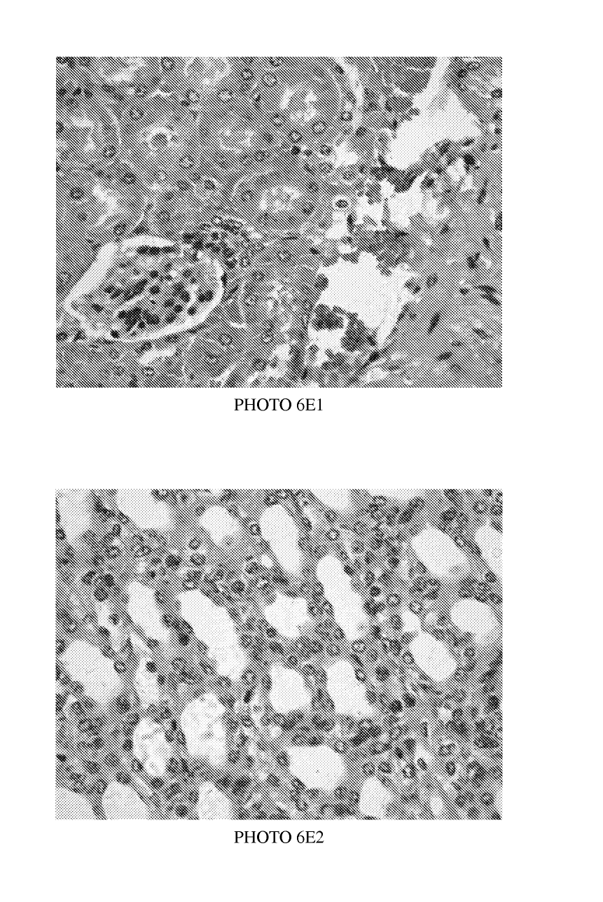

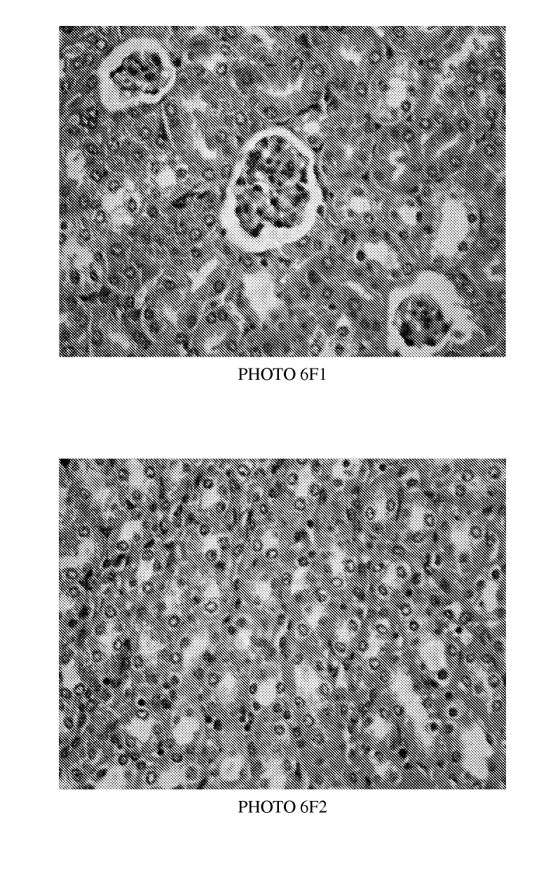

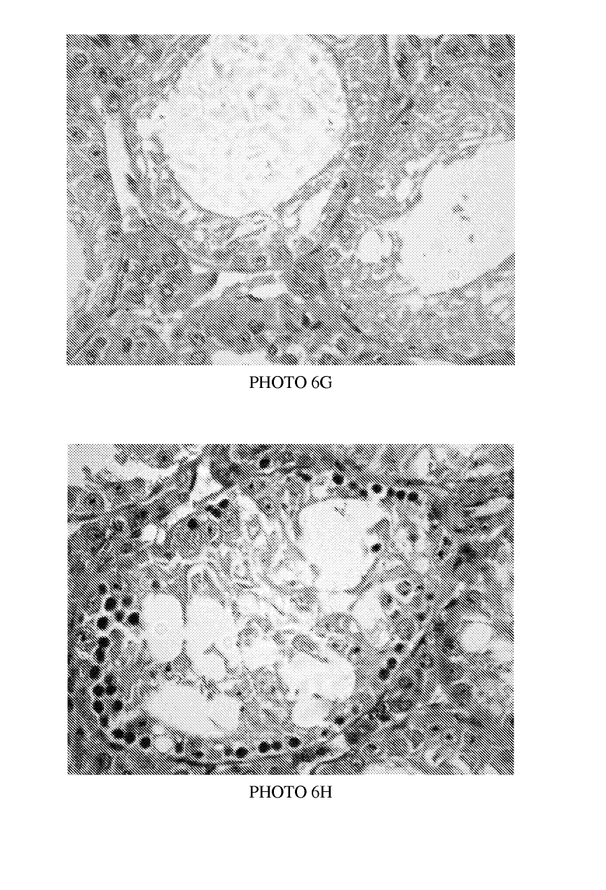

Expression of PprI protein in 293T cells was detected by Western blotting. The cells were washed twice with PBS and harvested by centrifugation at 5000 rpm at 4° C. for 5 min. The cells were then re-suspended in lysate buffer containing protease inhibitor cocktail (Calbiochem), the cell debris were removed by centrifugation. The supernatant was mixed with loading buffer, and the mixture was incubated at 95° C. for 5 minutes. Then 20 μl of the mixture was subjected to 12% SDS-polyacrylamide gel electrophoresis (PAGE). The proteins from PAGE were transferred onto a nitrocellulose (NC) filter. The blotting filter was blocked in fresh blocking buffer (0.1% Tween 20 in Tris-buffered saline, pH 7.4, containing 5% non-fat dried milk) and shaken at room temperature for 1.5 hours, and then incubated with shaking at 4° C. overnight in blocking buffer with HA-Tag mouse antibody (1:1000 dilution). Then the blotting filter was incubated with shaking at room temperature for 1 hour in the anti-mouse HRP-conjugated secondary antibody (1:5000 dilution) and washed again three times in TBST. Then the blotting filter was incubated with ECL substrate solution for 1 minute according to the manufacturer's instructions and visualized with exposure to X-ray film. The results show that the PprI protein can be detected in 293T cell transfected by the recombinant plasmid pCMV-HA-pprI and can not be detected in 293T cells transfected by the existing vector pCMV-HA. The molecular weight of the PprI protein is about 37 KD (FIG. 3).

### Example 3

### The Radioprotective Effects of the Recombinant pCMV-HA-pprI by In Vivo Electroporation on Lethally Irradiated Mice

1.1 Experimental Animals and Grouping

A pure breed of male BALB/c mice was used, provided by the Medical Laboratory Animal Center of Sichuan University. Their weights were 18±2 g. After about a week adjustment period of breeding, the mice were randomly divided into three groups: control group, radiation group and transgene group.

The animals of both the radiation and transgene groups were irradiated with neutrons or gamma rays. The irradiated mice were maintained continuously in a sterile room and 4 mice per group were sacrificed on days 1, 7, 14, 21 and 28 after irradiation for sampling and assay.

1.2 In Vivo Electroporation of the pprI Gene in Mice

The femoral anterior muscle of each mouse from the transgene group was injected 24 hours before irradiation with the pCMV-HA-pprI at a concentration of 50 μg/50 μl TE liquid, and then a pair of electrode needles were inserted, one either side of the DNA injection site, to deliver electric pulses. The recombinant plasmid was transferred into the muscle by in vivo electroporation at 8 electric pulses with electric field strength of 200 v/cm, duration of 20 ms and frequency of 1 Hz.

1.3 Preparation of the Model of Severe Acute Radiation Injury in Mice

1.3.1 Neutron Radiation

The total bodies of mice were irradiated with a K-400-DT neutron generator with a neutron mean energy of 14 MeV. Absorbed doses to the mice in the radiation groups were 0.2, 0.6, 1.0 and 2.0 Gy respectively, and that of the transgene group was 0.6 Gy.

1.3.2 γ-Ray Irradiation

The mice were total body irradiated with ⁶⁰Co γ-rays. The dose rate was 1 Gy/min, and the absorbed dose was 6.0 Gy.

1.4 Observation of the Radioprotective Effects of the pprI Gene by In Vivo Electroporation in Mice

1.4.1 Mortality of the Irradiated Mice

The irradiated mice were housed in a sterile room and given sterilised food and water. The mortality of the mice was monitored over the following 30 days.

Variations in the mortality of mice induced by different doses of neutron radiation are shown in Table 1.

TABLE 1
The mortality over 30 days among the mice irradiated by neutrons

| | Dose | | | Cumulative | Mortality |
|---|---|---|---|---|---|
| Group | (Gy) | n | Deaths/Days | deaths | (%) |
| radiation group | 0.2 | 8 | 0 | 0 | 0 |
| radiation group | 1.0 | 8 | 3/4; 3/5 | 6 | 75 |
| radiation group | 0.6 | 8 | 1/2; 1/9; 1/12 | 3 | 37.5 |
| radiation group | 2.0 | 20 | 17/4; 3/5 | 20 | 100 |
| transgene group | 0.6 | 8 | 1/13 | 1 | 12.5 |

As shown in Table 1, the greater the whole body neutron dose to the mice, the greater was the level of mortality. Severe acute radiation injury could be caused by 0.6 Gy of neutron radiation where 12.5% mortality in the transgene group was clearly lower than the 37.5% mortality recorded in the radiation-only 0.6 Gy group. The mice in the group exposed to 2.0 Gy all died on day 4 or 5 after the radiation.

1.4.2 Histopathological Examination of the Lung, Liver, Kidney and Testis from Irradiated Mice

Lung, liver, kidney and testis biopsy specimens from the mice in the radiation and transgene groups were fixed in formalin and embedded in paraffin. Sections were cut, placed on glass slides, and stained with Harris hematoxylin and eosin. Stained tissue sections were observed under a light microscope (Olympus). Coded slides were evaluated by a single expert pathologist to determine any histopathological changes.

Histopathological Changes of Lung:

In the radiation group (Photo 6A), the histopathological changes in the lung on day 28 after irradiation showed thickening of alveolar septa by edema, fibrous tissue, and a few inflammatory cells. The alveolus and its organizational structure were contorted and disorganized. In the irradiated transgene group (Photo 6B), there was a mild inflammatory reaction, and the histological recovery was remarkable with a return to normal structure on day 28 after irradiation.

Histopathological Changes of Liver:

In the radiation only group (Photo 6C) there were marked histopathological changes in the liver on day 21 after irradiation. These were mainly represented by mononuclear cell infiltration, congestion, an enlargement of the veins and sinusoids, hepatocellular degeneration, severe necrotic changes, break-up of nuclei, and general disorganized tissue structure. In irradiated transgene group (Photo 6D), there was more evidence of nuclear divisions and a mild increase in the number of Kupffer cells, and a full return to normal histological structure by day 21 after irradiation.

Histopathological Changes of Kidney:

In the radiation only group severe effects were observed on day 28 after irradiation. The glomerular capillaries exhibited vitriform degeneration, marked tubular dilation, hydropic degeneration in tubular epithelium, moderate congestion, and hemorrhage in the cortical and medulla part of the kidney (Fhotos 6E1 and 6E2)

The kidney had its normal structure, no hydropic degeneration, congestion and hemorrhage in the irradiated transgene group on day 28 after irradiation (Photos 6F1. and 6F2).

Histopathological Changes in Testis:

More shrinkage of tubules with cytoplasmic vacuolization and disappearance of spermatogonia were observed at day 28 after irradiation in radiation only treated mice (Photo G). In irradiated transgene animals, there was an increase in tubular diameter with the early spermatogonial population, and the testis had made an obvious recovery at day 28 (Photo H).

1.4.3 Observation of Leucocytes Count in Peripheral Blood

Blood was sampled from the orbital veins of the mice and put into tubes containing EDTA. Then 20 μl of blood was added to 0.38 ml of 2% of acetic acid, mixed, placed in a haemocytometer slide, left to stand for 5 min and then a leucocytes count was made in a microscope under low magnification.

The measured changes in total numbers of leucocytes are shown in Table 2.

TABLE 2
The changes of blood leucocytes in mice after irradiation ( X̅ ± S)

| | n | 1 d | 7 d | 14 d | 21 d | 28 d |
|---|---|---|---|---|---|---|
| control group (×10⁹/L) | 4 | 4.75 ± 0.39 | — | — | — | — |
| radiation group (×10⁹/L) | 4 | 0.93 ± 0.28** | 0.33 ± 0.13** | 0.50 ± 0.08** | 0.65 ± 0.20** | 1.75 ± 0.20** |
| transgene group (×10⁹/L) | 4 | 1.75 ± 0.31**^{##} | 0.70 ± 0.29** | 1.65 ± 0.34**^{##} | 2.23 ± 0.50**^{##} | 3.83 ± 1.02^{##} |

Note:
Compared with the control group
*P < 0.05,
**P < 0.01;
Compared with the radiation group,
^{#}P < 0.05,
^{##}P < 0.01.

As shown in Table 2, the leucocytes counts fell significantly in both the radiation only and transgene groups on day 1 after irradiation. The leucocytes reached a minimum on day 7 and began to recover on day 14. The leucocyte counts in the transgene group were statistically significantly higher than in the radiation only group on all sampling days (P<0.01), and it has recovered to normal on day 28 where the counts were consistent with those of the control group (P>0.05).

1.4.4 Observation of Lymphocytes Percentage from Peripheral Blood

Orbital vein samples were used to make blood smears which were stained with Wright's stain and scored with low magnification microscopy to measure the lymphocytes expressed as a percentage of all leucocytes.

The changes of lymphocytes percentage from peripheral blood at different times after neutron irradiation are shown in Table 3.

TABLE 3
The changes of blood lymphocytes percentage in mice after irradiation ( X̅ ± S)

| | n | 1 d | 7 d | 14 d | 21 d | 28 d |
|---|---|---|---|---|---|---|
| control group (%) | 4 | 44.00 ± 4.97 | — | — | — | — |
| radiation group (%) | 4 | 24.75 ± 2.98** | 21.25 ± 2.22** | 28.25 ± 1.5** | 26.00 ± 5.85** | 26.25 ± 3.5* |
| transgene group (%) | 4 | 24.00 ± 2.65** | 22.67 ± 3.79** | 32.00 ± 3.0** | 33.00 ± 6.00* | 33.3 ± 4.04* |

Note:
Compared with the control group
*P < 0.05,
**P < 0.01;
Compared with the radiation group,
^{#}P < 0.05,
^{##}P < 0.01.

As shown in Table 3, compared with the control group, the blood lymphocyte percentages reduced significantly in both the radiation only and transgene groups, with p values, as indicated, of <0.05 or 0.01. The lymphocyte percentages of both irradiated groups began to fall on day 1, reached a minimum on day 7 and began to recover slowly on day 14. The lymphocyte percentages in the transgene group were higher than in the radiation only group, but the differences did not reach statistical significance (P>0.05).

1.4.5 Assay of Apoptosis of Marrow Cells, Splenic and Thymic Lymphocytes in Mice.

Mouse spleens and thymuses were removed and teased with forceps in PBS to prepare single-cell suspensions. The marrow cavity of mouse femurs were flushed with PBS to prepare marrow cell suspension. Cell suspensions were centrifuged at 1500 rpm for 10 min, the supernatants discarded and the cell pellets resuspended in cold PBS. They were centrifuged again, supernatants discarded and the pellets suspended in 1×Annexin-V buffer solution, adjusting the concentration of the cells to 1×10⁶/ml. 100 μl of cell suspension was mixed with 5 μl of Alexa Fluor488 Annexin-V and 1 μl of PI (100 μg/ml) and incubated for 15 minutes at room temperature. Then 400 μl of 1×Annexin-V buffer solution was added, gently shaken and placed on ice. The samples were measured in a flow cytometer (Becton Dickenson Inc, USA) to detect apoptotic cells and the apoptosis rate (%) was expressed by a percentage of all cells present.

The changes in the apoptosis rates of marrow cells in mice are shown in Table 4.

TABLE 4
The changes of the apoptosis rates of marrow cells in mice after irradiation ( X̅ ± S)

| | n | 1 d | 7 d | 14 d | 21 d | 28 d |
|---|---|---|---|---|---|---|
| control group (%) | 4 | 5.31 ± 1.11 | — | — | — | — |
| radiation group (%) | 4 | 12.05 ± 3.33* | 34.98 ± 6.84** | 22.11 ± 3.49** | 18.26 ± 4.21** | 12.42 ± 1.28* |
| transgene group (%) | 4 | 5.81 ± 2.07^{##} | 14.50 ± 2.25*^{##} | 11.76 ± 3.35*^{#} | 11.70 ± 0.83*^{#} | 8.63 ± 2.75^{##} |

Note:
Compared with the control group
*P < 0.05,
**P < 0.01;
and compared with the radiation group,
^{#}P < 0.05,
^{##}P < 0.01.

As shown in Table 4, the apoptosis rate of marrow cell in the radiation only group increased significantly (P<0.01), while that in transgene group increased slightly but there was no statistical difference on day 1 after irradiation compared with the control group. The apoptosis rate in both irradiated groups increased significantly to the highest value on day 7. Their apoptosis rates decreased gradually on day 14. The apoptosis rate in the transgene group on day 28 had returned to normal and had no statistical difference compared with the control group. The apoptosis rate in the transgene group was always lower than that in the radiation group, and the differences were statistically significant or very significant (P<0.05 or 0.01).

The changes in the apoptosis rates of splenic lymphocytes in mice are shown in Table 5.

TABLE 5
The changes in the apoptosis rates of splenic lymphocytes in mice after irradiation ( X̅ ± S)

| | n | 1 d | 7 d | 14 d | 21 d | 28 d |
|---|---|---|---|---|---|---|
| control group (%) | 4 | 1.41 ± 0.22 | — | — | — | — |
| radiation group (%) | 4 | 2.61 ± 0.28** | 21.26 ± 5.71** | 12.53 ± 0.98** | 6.7 ± 1.78** | 4.41 ± 0.62** |
| transgene group (%) | 4 | 2.57 ± 0.01**^{##} | 6.64 ± 3.61**^{##} | 4.53 ± 1.01**^{##} | 4.14 ± 2.61**^{##} | 2.74 ± 0.55**^{##} |

Note:
Compared with the control group
*P < 0.05,
**P < 0.01;
Compared with the radiation group,
^{#}P < 0.05,
^{##}P < 0.01.

As shown in Table 5, the apoptosis rate of splenic lymphocytes in both the radiation only and transgene groups was increased very significantly (P<0.01) from day 1 to day 28 after irradiation compared with the control group. Their apoptosis rates maximised day 7. Compared with the radiation only group, the apoptosis rate in the transgene group was always very significantly lower (P<0.01).

The changes in the apoptosis rates of thymic lymphocytes are shown in Table 6.

TABLE 6
The changes in the apoptosis rates of thymic lymphocytes in mice after irradiation ( X̅ ± S)

| | n | 1 d | 7 d | 14 d | 21 d | 28 d |
|---|---|---|---|---|---|---|
| Control group (%) | 4 | 1.69 ± 0.25 | — | — | — | — |
| radiation group (%) | 4 | 18.67 ± 0.86** | 28.15 ± 4.51** | 16.28 ± 3.03** | 14.24 ± 4.33** | 13.55 ± 3.00* |
| transgene group (%) | 4 | 8.02 ± 4.31*^{#} | 15.02 ± 6.12*^{#} | 7.34 ± 0.63**^{##} | 5.84 ± 0.23**^{##} | 4.10 ± 4.46^{#} |

Note:
Compared with the control group
*P < 0.05,
**P < 0.01;
Compared with the radiation group,
^{#}P < 0.05,
^{##}P < 0.01.

Compared with the results for splenic lymphocytes in Table 5, the apoptosis rates for irradiated thymic lymphocytes shown in Table 6 were increased significantly. On the first day after irradiation, the apoptosis rates of thymic lymphocytes in the radiation only group started to clearly rise and reached a maximum on day 7. The rates began to fall on day 14, but still remained elevated until day 28. In the transgene group, the apoptosis rates of thymic lymphocytes was higher than that of the control group on day 1, began to rise on day 7, and then fell gradually, recovering to near normal levels on day 28. Over the duration of the entire experiment the apoptosis rates of thymic lymphocytes in the transgene group was always lower than that of the radiation only group, and the differences were significant or very significant (p<0.05 or 0.01). Note: The t test was used for testing differences between samples average in the above experimental data. It was performed with SAS 9.0 software.

The research results presented above prove that the recombinant vector pCMV-HA-pprI as a gene drug was transferred into the irradiated mice by in vivo electroporation. The drug effectively reduced the mouse mortality, and significantly decreased the number and degree of leucopaenia, and also significantly reduced the apoptosis rates of marrow cells, splenic and thymic lymphocytes, and clearly mitigated the range and extent of histopathological damage in the lung, liver, kidney and testis of irradiated mice, and promote cellular repair of these organs. It is concluded that the recombinant eukaryotic vector pCMV-HA-pprI encoding the pprI gene from D radiodurans R1 and its expressed PprI protein in vivo had significant prevention and treatment effects on severe acute radiation injury caused by neutron or gamma radiation

We claim:

1. A strain of E. coli DH5α containing a recombinant vector pCMV-HA-pprI.

2. A recombinant eukaryotic expression plasmid encoding the pprI gene of Deinococcus radiodurans R1, wherein the recombinant vector is pCMV-HA-pprI.

3. According to claim 2, wherein the method for constructing said pCMV-HA-pprI eukaryotic vector comprising:
(1) Obtaining pprI gene by PCR amplification using isolated total genomic DNA from Deinococcus radiodurans R1 as templates, using the primer (5′-ATGCCCAGTGCCAACGTCAGCCCCCCTT-3′) as upstream primer, the primer (5′-TCACTGTGCAGCGTCCTGCGGCTCGTCC-3′) as downstream primer, purifying and detecting and quantifying the PCR products, obtaining PCR product pprI gene;
(2) Ligating the PCR product pprI gene into a sub-cloning vector pGEM-T, then transferring the ligated product pGEM-T-pprI into E. coli DH5α, and picking out the positive clones after incubation of the bacteria, then extracting and sequencing the recombinant vector pGEM-T-pprI;
(3) Obtaining pprI fragment by PCR amplification using the recombinant vector pGEM-T-pprI as templates, using the primer (5′-TCGAATTCCCAGTGCCAACGTCAGCCCCCCTTGC-3′) as upstream primer, the primer (5′-TTCTCGAGTTTCACTGTGCAGCGTCCTGCGGCTC-3′) as downstream primer, obtaining the PCR product pprI fragment;
(4) Digesting the PCR product pprI fragment and the pCMV-HA vector by enzymes EcoRI and XhoI, obtaining the digested product pprI fragment digested pCMV-HA vector, then purifying and ligating the digested product pprI fragment into the digested pCMV-HA vector to construct the recombinant plasmid pCMV-HA-pprI.

4. A method for constructing the pCMV-HA-pprI eukaryotic vector capable of expressing the pprI gene of Deinococcus radiodurans R1, comprising:
(1) Cloning the pprI gene, comprising isolating Total genomic DNA from D. radiodurnas R1 as a template of PCR-amplification, the pprI gene clone primers was designed according to the promulgated genome sequence of D. radiodurans R1, wherein the forward primer is 5′-ATGCCCAGTGCCAACGTCAGCCCCCCTT-3, the reverse primer is 5′-TCACTGTGCAGCGTCCTGCGGCTCGTCC-3′, and wherein PCR was carried out with the above template and primers, and PCR products were purified using an agarose gel recovery and purification kit, detected and quantified by agarose gel electrophoresis;
(2) The construction of a sub-cloning vector pGEM-T, comprising ligating the PCR product pprI gene into a sub-cloning vector pGEM-T, the ligated product pGEM-T-pprI being transferred into E. coli DH5α, and the positive clones picked out after incubation of the bacteria, then the pGEM-T-pprI extracted and sequenced;
(3) The construction of recombinant plasmid pCMV-HA-pprI, comprising amplificating the recombinant vector pGEM-T-pprI using PCR with the two PCR primers, 5′-TCGAATTCCCAGTGCCAACGTCAGCCCCCCTTGC-3′ and 5′-TTCTCGAGTTTCACTGTGCAGCGTCCTGCGGCTC-3′, the underlined sequences being restriction sites of EcoRI and XhoI respectively, the PCR product digested by EcoRI and XhoI, and the fragment ligated into the pCMV-HA vector that had been predigested by the above enzymes, and wherein the recombinant plasmid pCMV-HA-pprI is transferred into E. coli DH5α, and cultured on LB plates solidified with 1.5% agar and supplemented with 100 μg/ml of ampicillin, the positive clones picked out after 12 h of incubation, and wherein PCR-amplification is carried out from different positive clones, the clones which contain recombinant plasmid pCMV-HA-pprI selected out by agarose gel electrophoresis, the recombinant plasmid pCMV-HA-pprI separated and sequenced, the positive bacterial clones having the correct sequence conserved.

5. An application of the recombinant vector pCMV-HA-pprI described in claim 2 to the preparation of the PprI protein as a drug for resistance to radiation injury.

6. A gene therapy drug for preventing and treating acute radiation injury, its character is the recombinant vector pCMV-HA-pprI described in claim 2.


SEQUENCE LISTING (2 sequences)

SEQ ID NO: 1 - DNA, 853 bp, Deinococcus radiodurans
tggggatggggagtggtacttctgctctaaagctgcggaattgtacccgcgggcccaccatgtacccatacgatgttccagattacgctcttatggccatggaggcccgaattcccagtgccaacgtcagccccccttgcccctctggggtaaggggcggggggatggggccaaaagctaaagctgaagcctccaagccccacccccaaatccctgttaagctcccattcgtgaccgcccccgacgccctcgccgccgccaaagccaggatgcgcgacctggcggcggcctacgtggcggccctgcccggacgcgacacccacagcctgatggcgggggtgcccggcgtagacctcaaattcatgccgctcggctggcgcgacggggcgttcgaccccgagcacaacgtcatcctcatcaactcggcggcccgccccgaacgccagcgcttcaccctcgcccacgaaatcgggcacgcgattttactcggcgacgacgacctgctctccgacatccacgacgcctacgagggcgagcggctcgaacaggtcatcgaaacgctgtgcaacgtggcggcggcggcgattttgatgcccgaacccgtcatcgcggaaatgctggaacgcttcggccccaccgggcgcgccctcgccgaactcgccaagcgggccgaagtcagcgcgtcgtcggcgctctacgccctgaccgagcagaccccggtgcccgtcatctacgcggtctgtgcgccgggcaagcctccgcgtgagcaggccgcaagcgacgaggacgctggcccaagcacagaaagagtcctgacggtccgcgccagcagctcgacgcggggcgtcaagta

SEQ ID NO: 2 - DNA, 845 bp, Deinococcus radiodurans
atttccaaaattaaagcattttttttcctgcattctagttgtggtttgtccaaactcatcaatgtatcttaccatgtctggatccccgcggccgcggtacctcgagtttcactgtgcagcgtcctgcggctcgtcccggtcggcctgctcgctgtccttgcggcccaggcgggcggggtcgaactcgaaactgacggccacgatgccgcgcgacgggtaggcgtccacctccgccttcattttccggcccgagcgaaagggcacgtagctttcctcgcgcacttccatgcccgtggcgagggcaagcgccgccgggtggtcggcgggtaccggcgtgccgctcgccagggtgtacttgacgccccgcgtcgagctgctggcgcggaccgtcaggactttttctgtgcttgggccagcgtcctcgtcgcttgcggcctgctcacgcggaggcttgcccggcgcacagaccgcgtagatgacgggcaccggggtctgctcggtcagggcgtagagcgccgacgacgcgctgacttcggcccgcttggcgagttcggcgagggcgcgcccggtggggccgaagcgttccagcatttccgcgatgacgggttcgggcatcaaaatcgccgccgccgccacgttgcacagcgtttcgatgacctgttcgagccgctcgccctcgtaggcgtcgtggatgtcggagagcaggtcgtcgtcgccgagtaaaatcgcgtgcccgatttcgtgggcgagggtgaagcgctggcgttcggggcgggccgccgagttgatgaggatgacgttgtgctcggggtcgaacgccccgtcgcgccagccgagcggcatga